(19) United States
(12) Patent Application Publication
(10) Pub. No.: US 2004/0166088 A1
(43) Pub. Date: Aug. 26, 2004

(54) Polymeric precursors of non-absorbable, in situ-forming hydrogels and applications thereof

(76) Inventor: Shalaby W. Shalaby, Anderson, SC (US)

Correspondence Address: LEIGH P. GREGORY, ATTORNEY AT LAW, PO BOX 168, CLEMSON, SC, 29633-0168, US

(21) Appl. No.: 10/758,357

(22) Filed: Jan. 15, 2004

Related U.S. Application Data
(60) Provisional application No. 60/440,195, filed on Jan. 15, 2003.

(51) Int. Cl. A61K 31/74

(52) U.S. Cl. 424/78.29

(57) ABSTRACT

The present invention is directed toward an injectable, single- or multiple-component polymeric liquid precursor of an in situ-forming, non-absorbable, flexible, and resilient hydrogel or semi-solid that can be used in non-surgical, minimally invasive treatment of herniated disc.

The present application claims the benefit of prior provisional application Serial No. 60/440/195, filed on Jan. 15, 2003.

### FIELD OF THE INVENTION

This invention relates to injectable polymeric precursors of an in situ-forming, non-absorbable hydrogel or semi-solid for replacing or augmenting the intervertebral discus nucleus pulposus.

### BACKGROUND TO THE INVENTION

Interest in liquid polymers that undergo physical transformation into three-dimensional gels or semi-solids upon exposure to certain environments has grown considerably over the past few years because of the unmet needs associated with contemporary pharmaceutical and biomedical applications. In an effort to satisfy one of the needs dealing with absorbable systems, the present inventor conceived and developed a number of absorbable hydrogel-forming, self-solvating liquid copolyesters that physically transform to three-dimensional gels or semi-solids upon contacting aqueous environments as disclosed in U.S. Pat. Nos. 5,612,052; 5,714,159; and 6,413,539. Cited in these patents are many pharma-ceutical and biomedical applications that call for transient absorbable materials with finite half-lives. However, growing demands for non-absorbable, biostable, easy-to-administer, biomedical implant precursors of physically or chemically crosslinked gels or semi-solids remain unmet. Accordingly, this invention deals with new polymeric precursors of non-absorbable and biostable precursors of hydrogels that can be easily introduced to specific biological sites using non-invasive means.

Among the unmet biomedical needs for novel hydrogels are those related to the degeneration of the spinal lumbar intervertebral discs. This can lead to loss of disc height, with a resulting decrease in segmental stability, as well as onset of lower back pain or neural deficits as a result of nerve root compression from a narrowing foramen. It is believed that 75 percent of the cases of chronic lower-back pain are associated with reduced mechanical functionality of the intervertebral disc (IVD) due to dehydration of the nucleus pulposus. This is a pulpy elastic substance comprising the central core of the IVD. Fibrous tissue and fibrocartilage form the disc outer rim (or annulus fibrosus). The nucleus pulposus (NP) consists of a matrix of fine collagen fibers, hydrophilic proteoglycan molecules, and up to 80 percent water. The annulus fibrosus has concentric cylindrical layers of fibrous collagen arrayed around the nucleus, like the layers of an onion skin. With age, the nucleus pulposus looses its resiliency. It may then be suddenly compressed by exertion or trauma and pushed through the annulus with fragments protruding into the spinal cord and pressing on the spinal nerves or spinal cord itself. Medically, this is referred to as herniated disc and is associated with severe back pain. Current treatment options for back pain associated with reduced disc functionality due to dehydration of the nucleus pulposus, range from conservative bed rest to highly invasive surgical interventions. The latter may entail spinal fusion and discectomy aimed at reducing pain, but not at restoring the disc function. Several investigators in the prior art attempted to replace the NP alone rather than the entire disc. This would result in a surgical technique that would offer a less invasive approach to pain relief while potentially restoring the functional biomechanics to the system. Thus, Q. B. Bao and P. A. Higham [U.S. Pat. No. 5,047,055 (1996)] have approached the NP replacement using semi-crystalline polyvinyl alcohol (PVA) implants, which undergo hydration to form a hydrogel. In addition to the need to use an invasive surgical procedure to introduce the PVA implant, its small crystallites melted, leading to reduction in the gel mechanical properties [S. R. Stauffer and N. A. Peppas, Polymer, 33, 3932 (1992)]. In an attempt to improve the performance of PVA, M. Marcolongo et al. [Sixth World Biomaterial Congress Transactions, 191 (2000)], using combinations of PVA and polyvinyl pyrrolidone (PVP), were unable to maintain the gel mass and elastic modulus to any practical extent for 30 days under the prevailing in vitro conditions. H. J. Wilke et al [Sixth World Biomaterial Congress Transactions, 190 (2000)] reported that a prosthetic disc nucleus (PDN) comprising a block copolymer of polyacrylamide and polyacrylonitrile encased in a woven polyethylene fabric has been implanted in humans and appears to exhibit promising initial results. However, all the NP replacements of the prior art required surgical intervention or were incapable of maintaining their initial gel mass and mechanical properties over a clinically relevant time period. Accordingly, this invention deals with polymeric precursors that can be injected non-invasively into the center of the annulus fibrosus to replace, or augment, compromised NP and exhibit expected biomechanical properties over clinically relevant time periods.

### SUMMARY OF THE INVENTION

Accordingly, the present invention is directed to an injectable polymeric composition which is a non-aqueous liquid that forms a non-absorbable hydrogel upon contact with an aqueous environment. In one embodiment, the non-aqueous liquid is a segmented/block copolymer comprising ether and peptide chain sequences. Preferably, such non-aqueous liquid is made by end-grafting an amine-terminated polyether with ε-caprolactam. In an alternative embodiment the non-aqueous liquid is a blend of a liquid succinic anhydride-bearing polyether and liquid diamine capable of an in situ reaction to form an amide-crosslinked network. For such embodiment it is preferred that the succinic anhydride-bearing polyether is made by a free-radical reaction of a polyether with maleic anhydride. In another embodiment the non-aqueous liquid is made by mixing a solution of succinic anhydride-bearing polyvinylpyrrolidone in liquid succinic anhydride-bearing polyalkylene glycol with a reactive liquid diamine or polyoxyalkylene diamine capable of forming an amide-crosslinked network. In yet another embodiment the non-aqueous liquid is a liquid urethane-interlinked polyether glycol capped with isocyanate end-groups. Alternatively, the non-aqueous liquid is a liquid polyether glycol capped with itaconic half-ester end-groups and a redox free-radical initiator system such as a combination of ascorbic acid and potassium persulfate.

In a still further embodiment the non-aqueous liquid is a dispersion of surface-maleated polypropylene microfibers and amine-terminated polyethylene glycol capable of forming a fiber-reinforced network in an aqueous environment, wherein the fibers are covalently linked to the polyethylene glycol-based matrix.

Preferred end-uses for the present non-aqueous liquid include a precursor for a hydrogel for augmenting the intervertebral disc nucleus pulposus, a precursor for a prosthetic intervertebral disc nucleus pulposus, and a precursor for a hydrogel for the treatment of herniated disc. In one embodiment the non-aqueous liquid further includes a cell-growth promoting agent selected from those known to accelerate tissue regeneration and site stabilization of a synthetic hydrogel prosthesis. It is preferred that the present non-aqueous liquid is prepared under aseptic conditions or terminally sterilized.

More specifically, the present invention deals primarily with injectable, single- or multiple-component polymeric precursors of in situ-forming, non-absorbable hydrogels or semi-solids that can be injected directly into the intervertebral disc to augment or replace the nucleus pulposus as a non-invasive or minimally invasive treatment of herniated discs. An aspect of this invention deals with an injectable precursor of a hydrogel prosthesis comprising a self-solvating, non-absorbable, non-aqueous liquid comprising a segmented/block copolymer comprising ether and peptide sequences, wherein the liquid precursor physically transforms to a hydrogel in the presence of water. Another aspect of the present invention relates to the preparation of the polymeric precursor of hydrogels or semi-solids by end-grafting amine-terminated polyethers with ε-caprolactam. In another aspect of the invention, the injectable polymeric precursor of the hydrogel prosthesis comprises a liquid succinic anhydride-bearing polyether and liquid alkane or polyoxyalkylene diamine capable of in situ reaction to form an amide-crosslinked network, wherein the anhydride-bearing polyether is made by reaction of maleic anhydride with the polyether and preferably in a solvent, such as toluene or dioxane in the presence of the free-radical initiator. Another aspect of this invention is directed to injectable polymeric liquid precursors of non-absorbable in situ-forming hydrogel or semi-solid made by mixing a solution of succinic anhydride-bearing polyvinylpyrrolidone in succinic anhydride-bearing, liquid polyalkylene glycol with a reactive liquid alkane or polyoxyalkylene diamine capable of forming an amide-crosslinked network. Another aspect of this invention deals with an injectable single component liquid polymeric hydrogel precursor comprising a liquid urethane-interlinked polyether glycol capped with isocyanate end-groups. Another aspect of the present invention relates to an injectable multiple-component liquid polymeric precursor of a hydrogel or semi-solid comprising a partially itaconized polylysine and an aqueous solution of a redox free-radical initiator system exemplified by a combination of ascorbic acid and potassium persulfate. Yet another aspect of this invention deals with injectable multiple-component polymeric liquid precursor of a hydrogel prosthesis comprising a liquid polyether glycol capped with itaconic half-ester end-groups and an aqueous solution of a redox free-radical initiator system exemplified by a combination of ascorbic acid and potassium persulfate. An additional aspect of the present invention pertains to an injectable liquid polymeric precursor of a fiber-reinforced hydrogel comprising a dispersion of surface-maleated polyethylene or polypropylene microfibers and amine-terminated polyethylene glycol capable of forming a fiber-reinforced network after mixing during injection and shortly after at the application site, wherein said fibers are covalently linked to the polyethylene glycol-based matrix. The injectable single- and/or multiple-component precursors of hydrogel, semi-solid or fiber-reinforced hydrogel described above can be used for augmenting or replacing the intervertebral disc nucleus pulposus as a non-invasive or minimally invasive treatment of herniated disc. The injectable single- and multiple-component precursors of the hydrogels, fiber-reinforced hydrogels and semi-solids noted above can be formulated to comprise a cell-growth promoting agent selected from those known to accelerate tissue regeneration and site stabilization of the synthetic hydrogel prosthesis. All forms of single- or multiple-component precursors of the hydrogels, fiber-reinforced hydrogels, or semi-solids described in this invention can be prepared under aseptic conditions or terminally sterilized using a suitable method, such as high energy radiation.

### DETAILED DESCRIPTION OF PREFERRED EMBODIMENTS

This invention deals primarily with single- or multiple-component liquid polymeric precursors of in situ-forming, non-absorbable, flexible, and resilient hydrogels or semi-solids. One aspect of this invention deals with injectable, water-insoluble, self-solvating, non-absorbable liquid segmented copolyamide made by end-grafting an amine-terminated hydrophilic polyether with a lactam, such as caprolactam, wherein the less hydrophilic polyamide segment is designed to be comiscible with the polyether segment in the absence of water. In the presence of an aqueous environment, the polyether segment absorbs most of the water and forces the less hydrophilic polyamide segments to aggregate, leading to a physically crosslinked hydrogel or semi-solid. The amine-terminated polyether can be based on a difunctional polyethylene glycol, difunctional block copolymer of polyethylene glycol-polypropylene glycol-polyethylene glycol (PEG-PPG-PEG) or amine-terminated polyoxyethylene diamine with branched chains.

Another aspect of this invention deals with in situ formation of a network through the reaction of polyethers having more than one succinic anhydride side groups per chain, with a low or high molecular weight diamine or polyoxyalkylene diamine. Specific cases of these systems include the following:

Case 1. Reaction of a liquid polyethylene glycol or its copolymer with polypropylene glycol carrying more than one succinic anhydride group per chain and preferably maleic half-ester end-groups, that is made by reacting the polyether sequences with maleic anhydride in the presence of a free-radical initiator (as described in U.S. patent application Ser. No. 10/693,361, filed on Oct. 24, 2003) with a liquid diamine, such as 1,4-butanediamine or low molecular weight polyoxyethylene diamine. The diamine then reacts with the anhydride group to form intermolecular amide crosslinks as part of the crosslinked hydrogel-forming network.

Case 2. Reaction of a liquid polyethylene glycol or poly(oxyethylene dimaleate) having succinic anhydride side groups as in Case 1 and a liquid polyoxyethylene diamine to produce a crosslinked, hydrogel-forming network as in Case 1.

Case 3. Reaction of liquid succinic anhydride-bearing polyether as in Case 1 with an aqueous solution of a polyamine, such as polylysine, for in situ formation of hydrogels.

Another aspect of this invention deals with liquid polyethylene glycol having two cyanoacrylate end groups, which undergo anionic polymerization upon injection into an aqueous environment to form a covalently crosslinked hydrogel. The cyanoacrylate-capped polyethylene glycol is prepared by reacting the polyethylene glycol with methyl or ethyl cyanoacrylate through acid-catalyzed transesterification as described in copending application, U.S. Ser. No. 10/300,079, filed on Oct. 20, 2002.

Another aspect of this invention deals with a crosslinked hydrogel-forming network made by reacting maleated polyvinylpyrrolidone microparticles dispersed or preferably dissolved in maleated liquid polyethylene glycol (prepared as described in copending application, U.S. patent Ser. No. 10/693,361, filed on Oct. 24, 2003), with a non-aqueous alkanediamine, or an aqueous solution of polylysine.

Another aspect of this invention deals with allowing maleated polypropylene (or polyethylene) microfibers (prepared by free-radical surface grafting with maleic anhydride using a free-radical initiator in toluene at 80-90° C. in which the polypropylene fibers were immersed) dispersion in liquid amine-terminated polyethylene glycol (i.e., polyoxyethylene diamine) during injection (using a special mixing device) and after residing in the biologic environment about the injection site to form a microfiber-reinforced, crosslinked hydrogel, wherein the microfibers are covalently linked at their surface to the polyoxyethylene diamine matrix through amide groups. This invention also deals with reacting polypropylene, or polyethylene, multifilament yarn with maleic anhydride in a dry organic liquid, such as toluene or dioxane, using a free-radical initiator, such as benzoyl peroxide or azo-bis-butyronitrile, to introduce succinic anhydride groups onto the surface of the polyolefin multifilament yam.

Another aspect of this invention addresses the use of a reaction product of polylysine with itaconic anhydride, or simply partially itaconized polylysine, as a precursor for in situ hydrogel formation, wherein a solution of the itaconic-bearing polylysine is allowed to crosslink under free-radical polymerization conditions, using a redox system, such as a combination of ascorbic acid and potassium persulfate. A specific aspect of this invention deals with using the hydrogel precursors described herein to inject directly into the intervertebral disc to produce a prosthetic nucleus pulposus. Another specific aspect of this invention deals with the use of hydrogel precursors herein in conjunction with a fiber construct to produce a prosthetic, intervertebral disc, with a nucleus and annulus-like components. Another aspect of this invention deals with the use of hydrogel precursors therein as injectable, soft prostheses to replace, or augment, compromised soft tissues, such as those of the breast and nucleus pulposus.

Another aspect of this invention deals with in situ covalent (through formation of covalent bonds) gelation/crosslinking of a liquid polyether (e.g., polyethylene glycol 400 or 600 and A-B-A block copolymer of polyethylene glycol-polypropylene glycol-polyethylene glycol having a molecular weight of 3300 Da) reacted with itaconic anhydride to form itaconic half-ester end-groups. The gelation/crosslinking can be achieved under free-radical conditions using a redox system, such as a combination of ascorbic acid and potassium persulfate. An aqueous solution of the redox system can be co-injected with the capped polyether (having itaconic half-ester at both terminals) directly into the vertebral disc to produce an in situ crosslinked hydrogel to augment or replace the nucleus pulposus. Another aspect of this invention deals with the aforementioned liquid polyethers interconnected by urethane linkage and capped with the isocyanate group. These can be prepared by reacting predried liquid polyether glycol, at 80-130° C., with an alkane diisocyanate (e.g., 1,6-hexane diisocyanate) using non-stoichiometric amounts of the reactants to insure interlinking as well as capping (e.g., a molar ratio of glycol/diisocyanate=0.6 to 0.9 and preferably 0.65 to 0.85). The urethane-interlinked, isocyanate-capped liquid polyether can be injected directly into the intervertebral disc. Upon exposure to the aqueous biological environment, part of the terminal isocyanate groups will be hydrolyzed to primary amine groups, which will react with the residual isocyanate groups to form urea interlinks leading to crosslinked network formation. A specific aspect of this invention deals with the use of the single- or multiple-component polymeric precursor of a hydrogel for direct injection using the proper delivery device (e.g., epidural needle or special spinal needle with or without a special attachment for delivering components of fiber-reinforced hydrogels) to insure facile delivery of the hydrogel precursor into the invertebral disc for treating herniated disc by augmenting or replacing the nucleus pulposus. Another aspect of this invention deals with using a hydrogel precursor that has been (1) prepared under aseptic conditions; (2) prepared by aseptic mixing of heat- or radiation-sterilized components; or (3) terminally sterilized by low- or high-energy radiation. A preferred aspect of this invention deals with a polymeric hydrogel precursor comprising one or more bioactive agent to improve its performance as a synthetic implant. For instance, an antimicrobial agent may be incorporated in the hydrogel precursor to prevent infection. A cell growth promoter, such as the ones used to accelerate tissue regeneration, may be incorporated into the hydrogel precursor. This may aid in accelerating tissue healing at the application site and allow for a timely mechanical stabilization of the prosthesis therein.

The invention may be further understood by reference to the following examples, which are provided for the purpose of representation and not to be construed as limiting the scope of the invention.

### EXAMPLE 1

### Synthesis of Liquid Urethane Interlinked Polyether Glycol Capped with Isocyanate Groups—General Method

A liquid polyether glycol (e.g., polyethylene glycol 400 and 600 and Pluronic 25-R4, Mₙ=3600 Da) is dried at 110° C. under reduced pressure (about 0.1 mm Hg) for 1 hour. An aliquot of the dried polyether glycol is mechanically mixed with diisocyanatoalkane (e.g., 1,6 hexane diisocyanate) using a glycol to diisocyanate molar ratio of less than one (e.g., 0.65 to 0.95) above room temperature (e.g., 30 to 50° C.) for about 10 minutes. The reaction temperature is raised above 70° C. (e.g., 80 to 130° C.). The reaction is continued until no significant change in the molecular weight (as determined by GPC) and isocyanate content (as determined by IR) could be detected over an additional period of 40 minutes. The product is cooled and poured under dry nitrogen atmosphere into a ready-for-use packaging form. A sample of the final product is analyzed for identify and composition (IR, NMR, elemental nitrogen analysis), equivalent weight (titration for isocyanate groups), and number and weight average molecular weight (GPC).

### EXAMPLE 2

### Preparation of Liquid Polyether Glycol Terminated with Itaconic Half-ester—General Method

A liquid polyether glycol (e.g., polyethylene glycol 400 and 600 and Pluronic 25-R4, Mₙ=3600 Da) is dried at 110° C. under reduced pressure (about 0.1 mm Hg) for 1 hour. An aliquot of the dried polyether glycol is mechanically mixed with itaconic anhydride, using a glycol to itaconic anhydride molar ratio of 0.5 or less (e.g., 0.5 to 0.35), at room temperature under a dry nitrogen atmosphere. The temperature mixing reactant is raised until the anhydride completely dissolved. A sample of this mixture is removed for analysis (GPC and IR). The temperature is then raised and maintained above 100° C. (e.g., 110-160° C.) for at least 1.5 hours (e.g., 1.5 to 5 hours) or until all the anhydride is consumed as determined by IR analysis. The final product is cooled and isolated. It is analyzed for molecular weight (GPC) and identity (IR) and composition (NMR).

### EXAMPLE 3

### Preparation of Liquid Succinic Anhydride-bearing Poly(oxyalkylene dimaleate) with Maleic Half-ester End-groups—General Method

A liquid polyalkylene glycol (e.g., polyethylene glycol 400, polyethylene glycol 600, or a block copolymer of polyethylene glycol and polypropylene glycol, such as Pluronic 25-R4) is sparged with oxygen-free nitrogen and then mixed with azo-bis-butyronitrile (ABIN) and maleic anhydride (MA) at the desired molar ratio of polyether/ABIN/MA (e.g., 1/2/3.9). The mixed reactants are heated, while stirring, at the minimum temperature (e.g., 40-65° C.) to achieve complete solution. The IR spectra of the solution is prepared to verify the semi-quantitatively the presence of characteristic anhydride and double-bond group frequency. The reaction is continued at the desired temperature (e.g., 65-110° C.) for the desired period of time (e.g., 2 to 6 hours) to complete incorporation of the maleic half-ester and succinic anhydride groups into the polyether chain. Infrared is used in monitoring the extent of the reaction.

### EXAMPLE 4

### Preparation of Injectable Succinic Anhydride-bearing Polyvinyl-pyrrolidine (PVP) in Liquid Succinic Anhydride-bearing Poly(oxyalkylene dimaleate)

An aliquot of liquid succinic anhydride-bearing poly(oxyalkylene dimaleate) (POADM, e.g., 50 g) is mixed with an aliquot of PVP (e.g., 5 to 20 g). The mixture was heated to form a viscous solution. This was transferred to a suitable device for co-injection with a liquid diamine or amine-terminated polyalkylene glycol (e.g., polyoxyethylene diamine).

Preferred embodiments of the invention have been described using specific terms and devices. The words and terms used are for illustrative purposes only. The words and terms are words and terms of description, rather than of limitation. It is to be understood that changes and variations may be made by those of ordinary skill art without departing from the spirit or scope of the invention, which is set forth in the following claims. In addition it should be understood that aspects of the various embodiments may be interchanged in whole or in part. Therefore, the spirit and scope of the appended claims should not be limited to descriptions and examples herein.

What is claimed is:

1. An injectable polymeric composition comprising a non-aqueous liquid that forms a non-absorbable hydrogel upon contact with an aqueous environment.

2. An injectable polymeric composition as set forth in claim 1 wherein the non-aqueous liquid comprises a segmented/block copolymer comprising ether and peptide chain sequences.

3. An injectable polymeric composition as set forth in claim 2 made by a process comprising the step of end-grafting an amine-terminated polyether with ε-caprolactam.

4. An injectable polymeric composition as set forth in claim 1 comprising a liquid succinic anhydride-bearing polyether and liquid diamine capable of an in situ reaction to form an amide-crosslinked network.

5. An injectable polymeric composition as set forth in claim 4 wherein the succinic anhydride-bearing polyether is made by a process comprising the step of a free-radical reaction of a polyether with maleic anhydride.

6. An injectable polymeric composition as set forth in claim 1 made by a process comprising the step of mixing a solution of succinic anhydride-bearing polyvinylpyrrolidone in liquid succinic anhydride-bearing polyalkylene glycol with a reactive liquid diamine or polyoxyalkylene diamine capable of forming an amide-crosslinked network.

7. An injectable polymeric composition as set forth in claim 1 comprising a liquid urethane-interlinked polyether glycol capped with isocyanate end-groups.

8. An injectable polymeric composition as set forth in claim 1 comprising a liquid polyether glycol capped with itaconic half-ester end-groups and a redox free-radical initiator system comprising a combination of ascorbic acid and potassium persulfate.

9. An injectable polymeric composition as set forth in claim 1 comprising a dispersion of surface-maleated polypropylene microfibers and amine-terminated polyethylene glycol capable of forming a fiber-reinforced network in an aqueous environment, wherein the fibers are covalently linked to the polyethylene glycol-based matrix.

11. An injectable polymeric composition as set forth in claim 1 as a precursor for a hydrogel for augmenting the intervertebral disc nucleus pulposus.

12. An injectable polymeric composition as set forth in claim 1 as a precursor for a prosthetic intervertebral disc nucleus pulposus.

13. An injectable polymeric composition as set forth in claim 1 as a precursor for a hydrogel for the treatment of herniated disc.

14. An injectable polymeric composition as set forth in claim 1 further comprising a cell-growth promoting agent selected from those known to accelerate tissue regeneration and site stabilization of a synthetic hydrogel prosthesis.

15. An injectable polymeric composition as set forth in claim 1 prepared under aseptic conditions or terminally sterilized.
